(19) United States
(12) Patent Application Publication
(10) Pub. No.: US 2024/0358449 A1
(43) Pub. Date: Oct. 31, 2024

(54) QUICK DISCONNECT OF A DISTAL SURGICAL ROBOTIC ARM

(71) Applicant: Asensus Surgical US, Inc., Durham, NC (US)

(72) Inventor: David John Bohle, II, Cary, NC (US)

(73) Assignee: Asensus Surgical US, Inc., Durham, NC (US)

(21) Appl. No.: 18/582,644

(22) Filed: Feb. 20, 2024

Related U.S. Application Data
(60) Provisional application No. 63/485,907, filed on Feb. 19, 2023.

(51) Int. Cl. A61B 34/30; A61B 90/50; B25J 17/00

(52) U.S. Cl. CPC A61B 34/30; A61B 90/50; B25J 17/00

(57) ABSTRACT

A surgical robotic manipulator includes a proximal assembly and a distal assembly, each of which is operable to move in a plurality of degrees of freedom. The distal assembly is removably attachable to the proximal assembly and secured in place by a primary fastening mechanism and a secondary fastening mechanism in the form of a quick release.

### RELATED APPLICATIONS

This application claims the benefit of U.S. Provisional Application No. 63/485,907, filed Feb. 19, 2023.

### TECHNICAL FIELD OF THE INVENTION

The present invention relates generally to the field of surgical devices and systems, and more particularly to the field of robotic manipulators for robot-assisted surgery.

### BACKGROUND

This application describes features that enable a rapid changeout of multiple axes of a robotic manipulator arm. Many robotic arms used in surgery include multiple axes, working together to accomplish a task, robotic surgery. It is beneficial for servicing and routine maintenance to have certain axes removed at once, together, to limit downtime or technician fatigue in the operating room or customer site. The configurations and methods described simplify the work of field service engineers and technicians who actively visit customer sites. They may be used to facilitate routine maintenance or necessary repairs. If the robotic manipulator is one having a variety of alternative, interchangeable distal ends suitable for different types of procedures, the configurations and methods described here can be used to allow service personnel to quickly change from one distal end to another.

The described concepts include a method for changing out the mechanical motors, encoders, sensors and other components, but also the electromechanical interconnects in that a specific electrical connector is used to pass all power and signals downstream to the changeable components. The connector mechanism floats to allow for some degree of misalignment, or normal and expected tolerance stack up.

### BRIEF DESCRIPTION OF THE DRAWINGS

FIGS. 1 through 15 illustrate a configuration in which the distal joints of a surgical robotic system may be separated from the proximal joints for servicing. More particularly:

FIG. 1 is a perspective view of a distal part of a robotic manipulator arm.

FIG. 2 is a perspective view of the robotic manipulator arm of FIG. 1.

FIG. 3 is a perspective view showing the proximal part of the distal subassembly coupled to the distal part of the boom assembly. A portion of the distal subassembly housing is shown as transparent.

FIG. 4 is similar to FIG. 3, but shows the proximal part of the distal subassembly de-coupled from the distal part of the boom assembly.

FIG. 5A is a side elevation view of the proximal part of the distal subassembly coupled to the distal part of the boom assembly. The distal subassembly is shown cut-away, allowing a cross-section view of the quick release mechanism and adjacent components to be visible.

FIG. 5B is a bottom perspective view of the proximal part of the distal subassembly coupled to the distal part of the boom assembly.

FIG. 6 is a distal end view of the boom assembly.

FIG. 7 is a cross-section view of the proximal part of the distal subassembly coupled to the distal part of the boom assembly.

FIG. 8 is a cross-section view of the proximal part of the distal subassembly de-coupled from the distal part of the boom assembly.

FIG. 9A is a cross-sectional side elevation view showing the circuit board of the distal sub-assembly.

FIG. 9B is a cross-section view of an O-ring of FIG. 9A.

FIG. 10 is a side elevation view of the proximal part of the distal subassembly of coupled to the distal part of the boom assembly in a second embodiment incorporating an alternative quick release mechanism. The distal subassembly is shown cut-away, allowing a cross-section view of the quick release mechanism and adjacent components to be visible.

FIG. 11 is a perspective view showing the proximal part of the distal subassembly of the FIG. 10 embodiment coupled to the distal part of the boom assembly. A portion of the boom assembly housing is shown as transparent.

FIG. 12 is similar to FIG. 11, but shows the proximal part of the distal subassembly de-coupled from the distal part of the boom assembly.

FIG. 13 is a perspective view of a robot-assisted surgical system comprising four robotic manipulator arms.

FIG. 14 is a perspective view of a robotic manipulator arm of FIG. 13 with the receiver/IDS and instrument assembly mounted to it.

FIG. 15 is a perspective view showing the IDS of FIG. 14 and the surgical instrument separated from the IDS.

### DETAILED DESCRIPTION

Co-pending and commonly owned U.S. application Ser. No. 18/582,601, (Attorney Docket: TRX-37000R), which is incorporated by reference, describes a surgical robotic manipulator having seven axes: a 3 DOF base assembly comprising a column and boom, and a 4 DOF “polso” mounted at the distal end of the boom. The present application describes quick disconnect configurations and methods in the context of replacing that polso of that arm. However, these same principles may be used on arms having other configurations, and on arms used for industrial applications or other purposes.

For the purpose of this description, the serviceable axes, or axes impacted by the principles described in this application, are referred to as the “distal axes” and J4-J7 (joint four through joint seven).

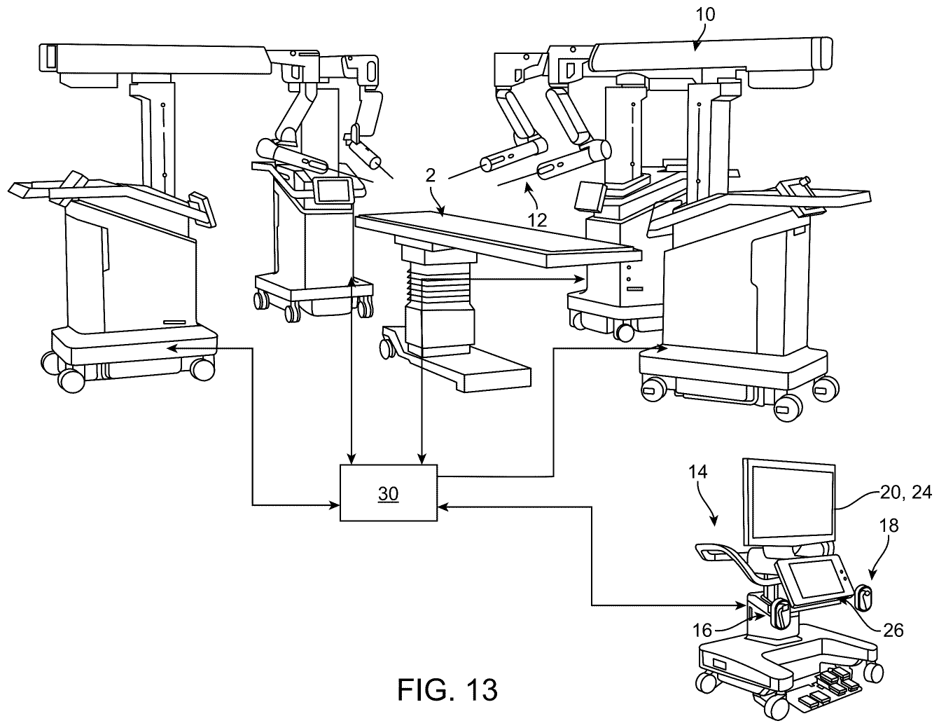

The robotic manipulator disclosed in U.S. Ser. No. 18/582,601 will be first described with reference to FIGS. 13-15 to facilitate an understanding of the inventive concepts described in the present application.

FIG. 13 shows robotic manipulators 10 disposed adjacent to a patient bed 2. Each manipulator 10 is configured to maneuver a surgical instrument 12 which has a distal end effector positionable in a patient body cavity. FIG. 13 shows four robotic manipulators, although in other configurations, the number of manipulators may differ.

A surgeon console 14 has two input devices such as handles 16, 18. The input devices are configured to be manipulated by a user to generate signals that are used to command motion of the robotic manipulators in multiple degrees of freedom in order to maneuver the instrument end effectors within the body cavity. The input devices may be mounted to linkages, gimbals, etc. equipped with sensors that generate signals corresponding to positions or movement of the input devices in manners known to those skilled in the art. In other embodiments, the input devices may take the form of handles that are tracked using a tracking system, such as an optical tracking system or an electromagnetic tracking system, either alone or in combination with other sensors within the handles, such as IMUs etc.

In use, a user selectively assigns the two handles 16, 18 to two of the robotic manipulators 10, allowing surgeon control of two of the surgical instruments 12 at any given time. To control a third one of the instruments disposed at the working site, one of the two handles 16, 18 may be operatively disengaged from one of the initial two instruments and then operatively paired with the third instrument, or another form of input may control the third instrument as described in the next paragraph.

One of the instruments 12 is a camera that captures images of the operative field in the body cavity. The camera may be moved by its corresponding robotic manipulator using input from a variety of types of input devices, including, without limitation, one of the handles 16, 18, additional controls on the console, a foot pedal, an eye tracker 20, voice controller, etc. The console may also include a display or monitor 24 configured to display the images captured by the camera, and for optionally displaying system information, patient information, etc. An auxiliary display 26, which may be a touch screen display, can further facilitate interactions with the system.

The surgical system allows the operating room staff to remove and replace the surgical instrument 12 carried by a robotic manipulator 10, based on the surgical need. When an instrument exchange is necessary, surgical personnel remove an instrument from a manipulator arm and replace it with another.

As discussed, manipulation of the input devices 16, 18 results in signals that are processed by the system to generate instructions for commanding motion of the manipulators in order to move the instruments in multiple degrees of freedom and to, as appropriate, control operation of electromechanical actuators/motors that drive instrument functions such as articulation, bending, and/or actuation of the instrument end effectors. One or more control units 30 are operationally connected to the robotic arms and to the user interface. The control units receive user input that is generated as a result of movement of the input devices, and generates commands for the robotic arms to manipulate the surgical instruments so that the surgical instruments are positioned and oriented in accordance with the input provided by the user.

Sensors in the robotic manipulators determine the forces that are being applied to the patient by the robotic surgical tools during use. U.S. Pat. No. 9,855,662, entitled Force Estimation for a Minimally Invasive Robotic Surgery System, which is incorporated herein by reference, describes a method by which input from a 6 DOF force/torque sensor on the robotic manipulator is used to determine the RCM about which the surgical instrument should be pivoted, which corresponds to the location of the incision along the instrument shaft. Motion of the robotic manipulator is thus algorithmically controlled to constrain the motion such that the instrument pivots relative to the RCM. In the presently disclosed embodiments, a sensor of this type may be optionally positioned just proximal to the instrument drive system 104. It should be understood that use of the 6 DOF force/torque sensor to determine the RCM is desirable, but is not essential for practice of the present invention. Other methods for determining the location of the RCM, including those discussed in the Background, may be used in the presently-described manipulator arm in lieu of methods using the 6 DOF force/torque sensor.

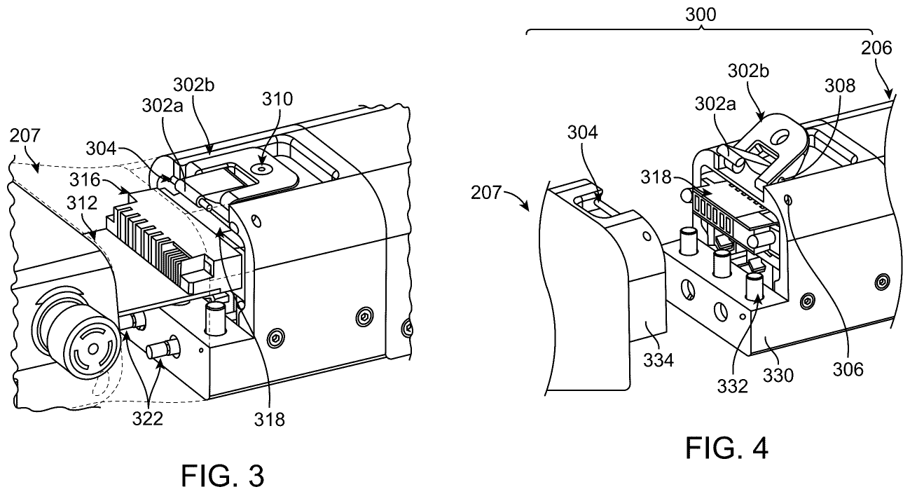

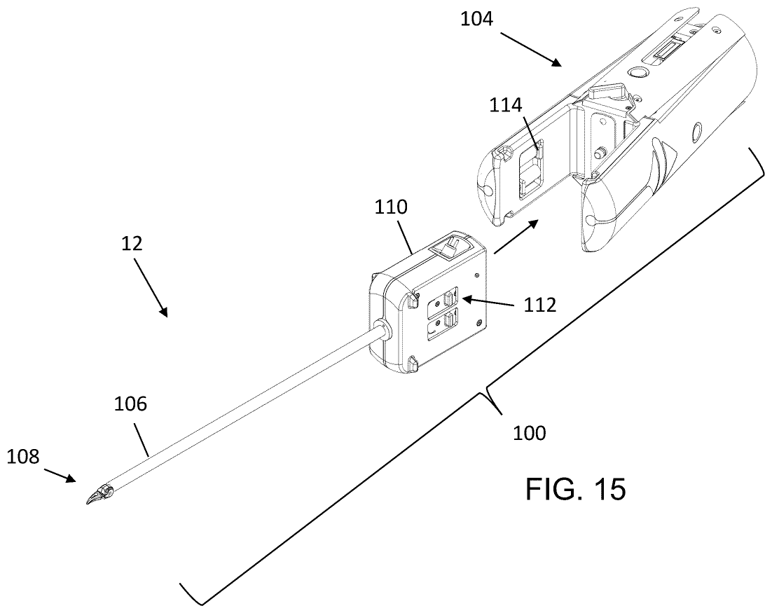

Referring to FIGS. 15, positioned at the distal end of each manipulator arm is a receiver 104, which may also be referred to as an instrument drive assembly (IDS). A different surgical instrument 12 is removably mountable to each IDS. As best seen in FIG. 3, each instrument 12 includes an elongate shaft 106, which is preferably rigid but which may be flexible or partially flexible in alternative systems. An end effector 108 is positioned at the distal end of shaft 106, and a base assembly or adapter assembly 110 is at the proximal end.

Instrument and IDS configurations suitable for use with the disclosed inventions will next be described, but it should be understood that these are given by way of example only. The disclosed manipulator may be used with various configurations of instruments and instrument drive systems. More particularly, while the receiver/IDS described here is configured to drive pitch and jaw motion of an articulated surgical instrument, in alternative embodiments the receiver/IDS may have less functionality. In some alternative configurations, it may serve simply to receive an instrument and to drive jaw open/close operations. In other configurations, it may be configured, along with the instrument, to actuate a roll function of the instrument tip relative to the shaft of the instrument.

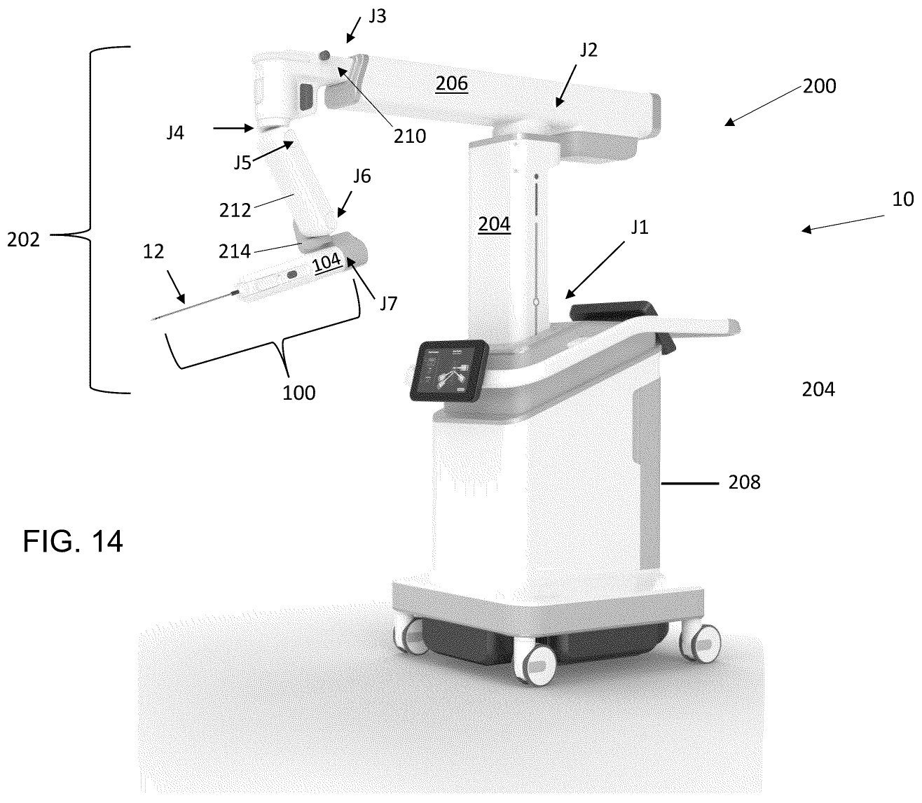

The instrument depicted in the drawings is the type described in Applicant's commonly-owned co-pending application published as US 2020/0375680, entitled Articulating Surgical Instrument, which is incorporated herein by reference. It makes use of four drive cables two of which terminate at one of the jaw members and the other two of which terminate at the other jaw member. This can be two cables looped at the end effector (so each of the two free ends of each cable loop is at the proximal end) or it can be four individual cables. As described in the co-pending application, the tension on the cables is varied in different combinations to effect pitch and yaw motion of the jaw members and jaw open-close functions. Other instruments useful with the system will have other numbers of cables, with the specific number dictated by the instrument functions, the degrees of freedom of the instrument and the specific configuration of the actuation components of the instrument. Note that in this description the terms “tendon,” “wire,” and “cable” are used broadly to encompass any type of tendon that can be used for the described purpose. The surgical instrument's drive cables extend from the end effector 108 through the shaft 106 (FIG. 14) and extend into the adapter assembly 110 where they are coupled to mechanical actuators. A more detailed description is given in Applicant's co-pending application published as US 2021/169595, which is incorporated herein by reference, but a general configuration of these actuators with respect to the adapter assembly will be provided here.

The adapter assembly 110 (which will also be referred to as the “adapter”) may include an enclosed or partially enclosed structure such as a housing or box, or it may be a frame or plate. The exemplary adapter 110 shown in the drawings includes mechanical input actuators 112 exposed to the exterior of the surgical instrument 102. In FIG. 15, two mechanical input actuators 112 are exposed at a first lateral face of the adapter 110. A second two mechanical input actuators 112 (not visible in FIG. 15) may be exposed at the second, opposite, lateral face of the adapter 110, preferably but optionally in a configuration identical or similar to the configuration shown in FIG. 3.

Each of the mechanical input actuators 112 is moveable relative to the adapter 110 between first and second positions. In the specific configuration shown in the drawings, the actuators are longitudinally moveable relative to the housing between a first (more distal) position and a second (more proximal) position such as that shown in FIG. 3. The direction of motion, however, is not required to be longitudinal and can extend in any direction.

In this configuration, the adapter thus has four drive inputs, one for each of the input actuators 112, exposed to its exterior. The illustrated adapter has two parallel planar faces, with two of these inputs positioned on each of the faces. While it may be preferred to include the inputs on opposite sides of the proximal body, other arrangements of inputs on multiple faces of the proximal body can instead be used. Each of these configurations advantageously arranges the drive inputs to maximize the distance between control inputs, minimizing stresses in the sterile drape that, in use, is positioned between the proximal body and the receiver 104. Co-pending US 2021/169595 includes further description of the adapter shown in FIG. 15.

The IDS 104 at the end of each manipulator 10 has an open position (shown in FIG. 15) in which it removably receives the adapter 110 of a corresponding instrument 12, to form an assembly 100. After the adapter 110 is placed within the IDS, the IDS is moved to the closed position shown in FIG. 14, capturing the adapter 110. In this position, the drive inputs 112 of the adapter can engage with corresponding drive outputs 114 of the IDS. As described in detail in co-pending US 2021/169595, user input at the input devices 16, 18 commanding jaw open-close, pitch or yaw articulation etc. of the instrument causes electromechanical actuators in the IDS to move the drive outputs 114. The motion of those drive outputs moves corresponding ones of the adapter's drive inputs 112, altering tension on the instrument's drive cables in a manner that causes the desired motion at the instrument's end effector.

The manipulator kinematic structure includes three primary components. Each component plays a different role in the overall instrument motion which, during laparoscopic motion of the instrument 106, must occur while algorithmically constraining the motion at the fulcrum/RCM F, which corresponds to the incision site, the position of which is calculated as discussed above. The first two are integral components of the manipulator: a base structure 200, formed of the proximal joints J1, J2 and J3 (discussed below), and a distal structure 202, formed of the distal joints J4-J7 (discussed below). The base structure functions primarily to place the distal structure above the surgical site by means of translation motions, whereas the primary purpose of the distal structure is to produce additional three DOF changes in the orientation of the instrument tip. Thus, during operation of the manipulator, motion of the proximal joints of the base structure 200 primarily governs position of the distal structure in three degrees of freedom, whereas motion of the distal joints 202 primarily governs orientation of the instrument tip in 3+, and preferably 4+ degrees of freedom. These two parts are discussed in further detail below. The third part is the instrument structure which in preferred embodiments is the IDS 104 or an alternative component which operates to control the instrument's distal joint(s). This part may optionally also include joints to allow for off-axis instrument placement.

The joints of base structure 200 and distal structure 202 will be further discussed in connection with FIG. 14. The base 200 structure is configured to reposition the distal structure 202 in 3 DOFs using a sequence of prismatic-revolute-prismatic joints, also referred to herein as the proximal joints. These motor-driven proximal joints are identified in the drawings as J1, J2 and J3, with JI being a prismatic joint, J2 being a revolute joint, and J3 being a prismatic joint. The prismatic joints may alternatively be referred to as rectilinear translation joints. As can be seen in the drawing, these joints are arranged in sequence, without intervening joints that would influence positioning of the distal structure 202.

The robotic manipulator includes a vertical column assembly 204 and a horizontal boom assembly 206. The vertical column assembly 204 preferably extends from a cart 208 having wheels that allow the robotic manipulator to be repositioned within the operating room by rolling it across the floor. Boom assembly 206 supports the distal structure 202 at its distal end.

Joint JI extends and retracts the vertical column assembly 204 relative to the cart 208 along a vertical axis. This motion functions to raise and lower the boom assembly 206. J1 may be configured to permit between 650-800 mm of extension of the vertical column assembly 204, and more preferably between 700-750 mm.

Joint J2 pivots the boom assembly 206 relative to a vertical axis of the column assembly 204. In preferred embodiments, J2 is configured to permit up to 180 degrees of rotation (90 degrees in each direction). Joint J3 extends and retracts a part 210 of boom assembly 206 along a horizontal axis. J3 may be configured to permit between 650-800 mm of extension of the boom assembly 206, and more preferably between 700-750 mm.

The distal structure 202 is configured to produce changes in orientation of the instrument tip in an additional 3DOFs. The distal structure has at least four motor-driven joints, also referred to as the distal joints, arranged to generate redundancy in joint space. The locations of the joints allow for uniform dexterity and minimize effect of singularities and joint limits along the workspace. The architecture of distal structure 202 includes a first link 212 coupled to the distal end of the boom assembly 206, and a second 214 at the distal end of the first link 212. Second link 214 supports the IDS 104, as well as the force/torque sensor (not shown), which is situated just proximal to the IDS and distal to joint J7 (discussed below). As discussed above, the feedback from the force/torque sensor is used in the control of motion of the arm 10 to ensure that movement of the instrument is algorithmically constrained about the fulcrum at the incision site.

It is useful to describe the distal joints with respect to a plane P containing the longitudinal axis of the instrument shaft 106 (preferably a vertical plane). The preferred kinematic configuration of the distal structure 202 includes four motor-driven distal joints identified as J4-J7. J4 is a revolute joint disposed between the distal end of the boom assembly 206 and link 212 (labeled in FIG. 14). Actuation of J4 produces rotation of the link 212, and thus distal structure 202, about a vertical axis Y, the axis of joint J, which lies within the plane P. Said another way, when joint J4 is driven, the plane P containing the longitudinal axis of the instrument shaft rotates about a vertical axis. In operation, J4 is preferably the primary contributor of yaw motion of the instrument shaft. A yaw axis of the manipulator is the axis about which rotation of the plane P containing the instrument shaft occurs. Yaw axis Y intersects the longitudinal axis of the instrument shaft and, preferably during operation of the arm, intersects the RCM defined at the fulcrum, in order to optimize the dexterity of the system.

In a preferred embodiment, J4 is configured for unrestricted rotation, although in alternative embodiments it may be configured for less than unrestricted rotation, such as 360 degree or 540 degree rotation.

Distally adjacent to J4 is a series of revolute joints, J5 and J6, which are coplanar and thus have parallel axes of rotation. These joints are located at opposite ends of link 212. The rotational axes of J5 and J6 have an orientation that is perpendicular to the plane P containing the yaw axis and the longitudinal axis of the instrument shaft.

J5 and J6 operate to move the longitudinal axis of the instrument within plane P. Operation of these joints can be used to effectuate pitch motion of the instrument shaft. During pitch motion of the instrument shaft 106 through operation of J5 and/or J6, the longitudinal axis of the instrument shaft remains within plane P. When J5 is actuated, it causes planar pitch motion of link 212 about the J5 axis, and when J6 is actuated it pivots the distal link 214 about the J6 axis, which is parallel to the J5 axis. Operation of J5 and J6 can thus be used to generate pitch motion of the distal link 214 (and consequently the instrument mounted to the link 212 via the IDS). Operational ranges for J5 and J6 are +/−110 degrees, and, more preferably, +/−90 degrees. It should be noted with regard to the joint ranges given in this application that operational ranges used in practice may be less than the joints' functional limits. In some cases, the control algorithms may artificially reduce the range of motion of all or certain joints. This may be done, for example, to accommodate smooth motion of other joints. Such operational limits may be configuration dependent, varying based on where the instrument or the relevant joint is in the workspace.

The final joint in the series of distal joints is revolute joint J7. Operation of J7 produces roll motion of the instrument shaft about an axis that is coincident with the longitudinal axis of the instrument shaft. The rotational axis of J7 has an orientation that is perpendicular to the orientations of pivot axes of each of J5 and J6, but it does not intersect with them. J7 may be configured for unrestricted rotation or for less than unrestricted rotation, such as 360 degree or 540 degree rotation. When J7 is operated, the force torque sensor that is just proximal to the IDS, as well as the IDS and the instrument, are caused to rotate.

In some cases, the arm may be provided without J7. This might be suitable in cases, for example, where the surgical instrument is one that has an end effector configured to axially roll relative to the instrument shaft using input from the IDS or actuators carried by the instrument adapter.

As illustrated in the prior referenced application, J4-J7 are part of a common sub-assembly. The goal is to have these four axes removed at once, together, with minimal time to limit a service disruption to the operating room or customer site.

More specifically, distal subassembly 207 which includes J4-J7 is removably connected to boom assembly 206. The connection between the distal sub-assembly and the boom assembly includes a primary fastening mechanism and a second fastening mechanism. The secondary fastening mechanism provides a safety lock that retains the distal subassembly 207 to the boom assembly 206 even after the primary fastening mechanism has been released.

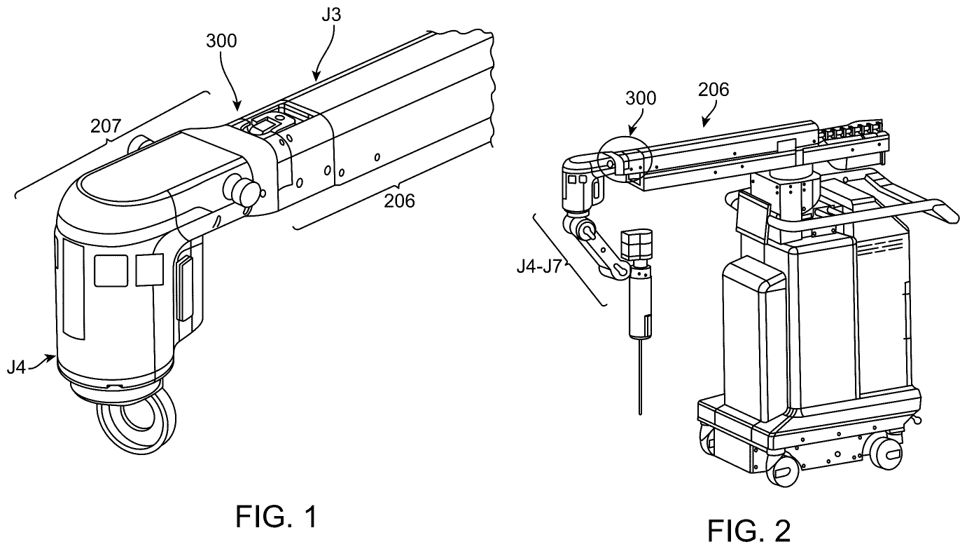

The first disclosed secondary fastening mechanism, which will be described with respect to FIGS. 1 through 9B, uses what is known as a “suitcase latch” or over-center mechanism to pull one entity towards another and fasten the distal end of the boom assembly 206 to the proximal end of the distal subassembly 207. The second disclosed secondary fastening mechanism uses a spring plunger to provide a safety lock after the primary fastening mechanism is released. Note that for both concepts shown, the primary fastening mechanism is a set of socket head cap screws mechanically fastening the two halves of the system together.

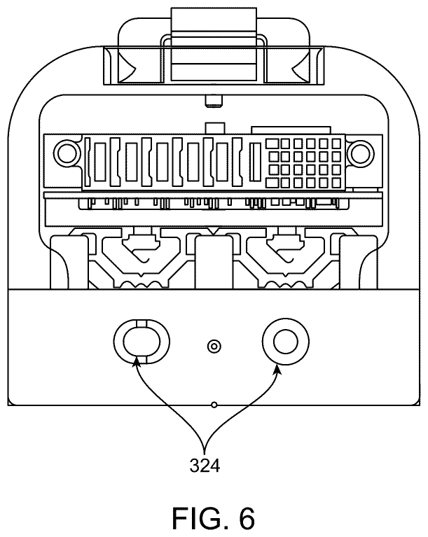

Use of both is performed according to the same general process which is as follows.
1. The robotic manipulator is powered off and placed in a state where it is ready for servicing
2. The Field Service Engineer (FSE) or Technician (Tech) removes the safety hardware
3. The FSE or Tech disengages the quick disconnect, causing the J4-J7 distal unit (distal subassembly 207) to become loose. The FSE or Tech maintains control of the distal subassembly 207-to prevent it from falling or becoming damaged.
4. The FSE or Tech fully removes the distal subassembly 207 and sets it to the side.
5. The FSE or Tech replaces the distal subassembly 207 with the FRU (Field Replaceable Unit)
6. The key step in the process is lining up the pre-alignment pins so the mechanism couples properly. As will be discussed in the description of FIGS. 6 through 8, the pre-alignment pins are long, stainless steel pins that engage with the mating half (proximal robot side) before any electrical contact is made. This ensures easy coupling and no chance of damage during coupling.
7. The FSE or Tech engages the secondary fastening mechanism back to the locked condition, thereby coupling the FRU of the distal subassembly 207 to the boom assembly 206.
8. At this point the FSE or Tech could let go of the FRU and it will hang securely fastened in space.
9. The FSE or Tech re-installs the safety bolts and verifies the torque.
10. The operation for quick change is complete, power may be restored, and normal operation of the robot can resume.

Two embodiments of quick disconnect subassemblies are described in this application: the suitcase clamp configuration and the spring plunger configuration. Below is a description of the parts of each subassembly:

### Suitcase Clamp Embodiment

In FIGS. 1 and 2, the distal subassembly 207 (which includes J4-J7) is shown as the distal end of the robot, coupled to boom assembly 206 which is the distal component of the J1-J3 assembly. In this embodiment, the secondary fastening mechanism 300 is in the form of a suitcase clamp, and includes a first portion 302 on the J1-J3 assembly 206 and a second portion 304 on the distal subassembly 207. The first portion 302 includes a hook 302a and a finger latch 302b, and the second portion 304 comprises a pin. The latch and hook engage the pin on the J1-3 side of the robot. This interface is referred to herein as the quick release.

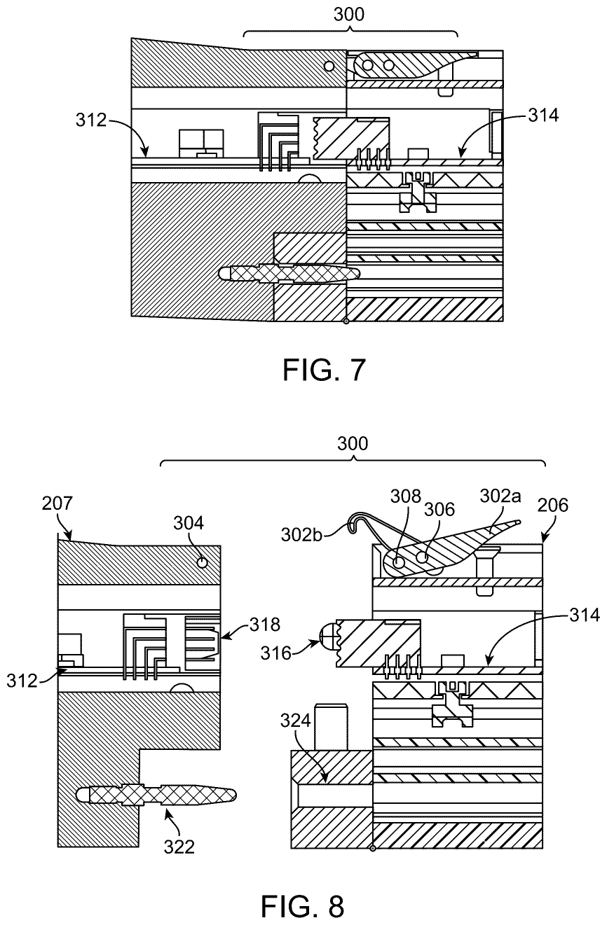

More specifically, FIGS. 3 and 7 show the quick release in the closed and latched position, and FIGS. 4 and 8 show the suitcase clamp in the opened position. As best seen in FIGS. 4 and 8, the finger latch 302a is pivotally mounted to the surrounding housing of the boom assembly 206 at a pivot 306, and the hook 302a is pivotally mounted to the finger latch 302a at pivot 308. To engage the quick release, the hook 302a is hooked over the pin 304, and then finger latch is pivoted to the closed position shown in FIG. 3 to latch the quick release. To further secure the quick release, a fastener such as safety bolt 310 may be installed to secure the finger latch 302a to the housing of the boom assembly 206. See FIG. 3.

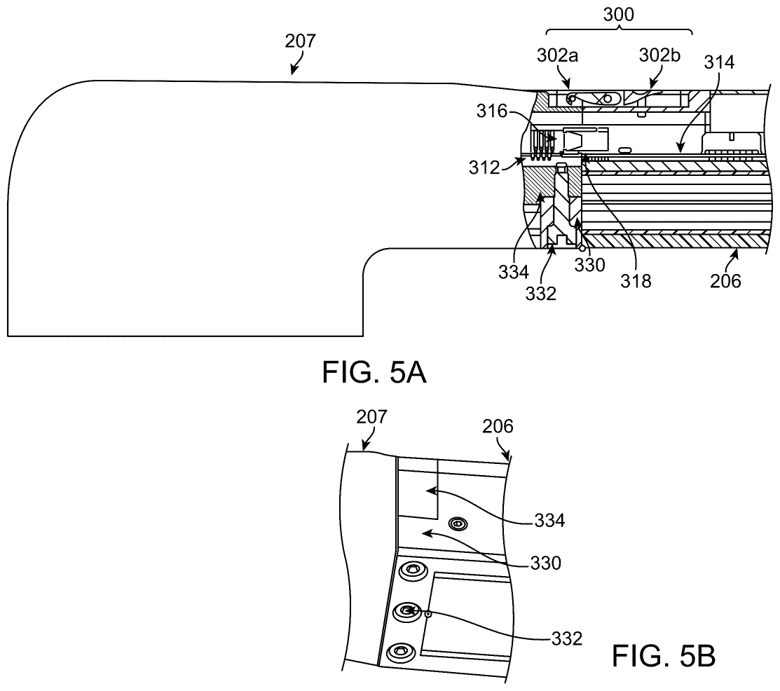

Referring to FIGS. 5 and 7, each of the distal subassembly 207 and the boom assembly 206 houses a circuit board 312, 314. Circuit board 312 is provided with an electrical connector 316, and circuit board 314 is provided with an electrical connector 318. These mate together as shown in FIGS. 3, 5, and 7, forming a butt joint, and connect all electrical contacts. This allows power and signal for J4-J7 to be passed from the base structure 200 to the distal structure 202.

While the connectors 316, 318 on the circuit boards have plastic alignment features, the significant mass of the distal subassembly necessitates use of a pre-alignment feature. Stainless steel alignment pins 322 are used for this purpose. Referring to FIG. 8, the alignment pins are fastened in the distal subassembly (J4-J7 end) and, during assembly of the distal subassembly to the boom assembly, the alignment pins engage metallic bores 324 in the boom assembly (J1-J3 end). As best shown in FIG. 6, one of the bores 324 is ovalized so as to not overdefine the system. Additionally, the openings to the bores 324 may be chamfered to facilitate alignment pin insertion.

During mounting of the distal subassembly to the boom assembly, the alignment pins 322 are inserted into the bores 324 before any electrical contact is made using connectors 316, 318. As best seen in FIG. 8, this is facilitated by the positions of the relevant components. As the distal subassembly 207 is moved into engagement with the boom assembly 206, the alignment pins 322 will enter the corresponding bores 324 before the electrical connectors 316, 318 come together. This prevents damage to the electrical contacts and allows for the FSE or tech to easily re-engage the quick release to the lock condition.

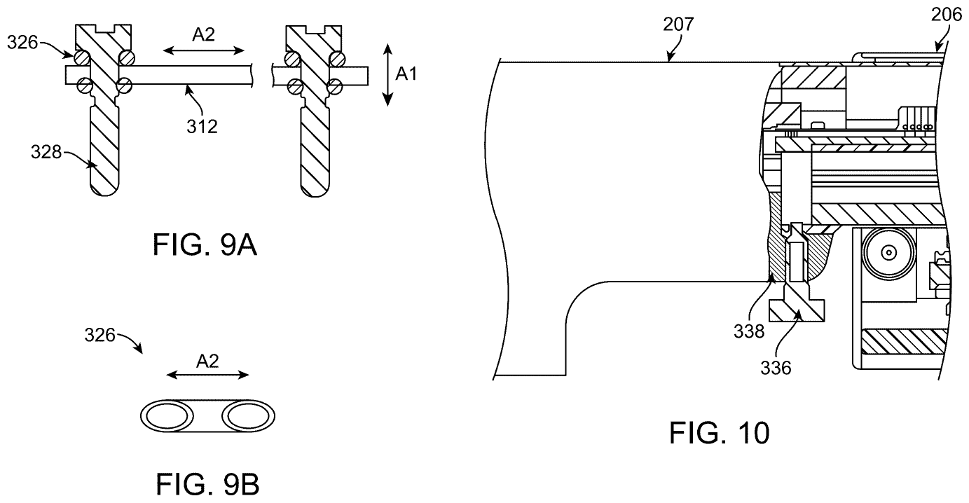

Since the assembly of the distal subassembly and the boom assembly creates a system that is fully constrained, tolerances dictate having some element of compliance between the circuit boards 312, 314. In other words, although there is a rigid lock between mechanical components, float is needed to ensure fragile circuit board components are not damaged. This is accomplished by having the circuit board in the distal subassembly assembly float on elastomer elements. This is achieved using compliance from O-rings 326, which allow the circuit board to float up and down, as indicated by arrow A1, and laterally, as indicated by arrow A2. As shown in FIG. 9A, two O-rings may be used on each fastener 328 that retains a circuit board within the distal subassembly or boom assembly. The O-rings have an oval-shaped cross-section as depicted in FIG. 9B, with the long-axis of the oval extending laterally, to facilitate float in the lateral direction A2. As shown, for each fastener, there may be one O-ring 326 positioned above the circuit board and another positioned below it. In alternative embodiments, however, springs, or other flexible elements could be used.

The figures show the use of three safety bolts as the primary fastening mechanism. These bolts are used to retain the distal subassembly in place during dynamic movement or operation. In the specific embodiment that is shown, the boom assembly 206 includes a distally-protruding member 330 (FIG. 4) with bores that receive the bolts 332. Distal subassembly 207 includes a proximally-protruding member 334 that, when the arm is assembled, seats on the distally-protruding member 330 as shown in FIGS. 5A and 5B. Once the protruding members are brought together in this manner, their corresponding bores are aligned, allowing for installation of the bolts 332. Note that in this embodiment, the bores 324 that receive alignment pins 332 are also formed in the distally protruding member 330 (see, e.g. FIG. 8).

### Spring Plunger Embodiment

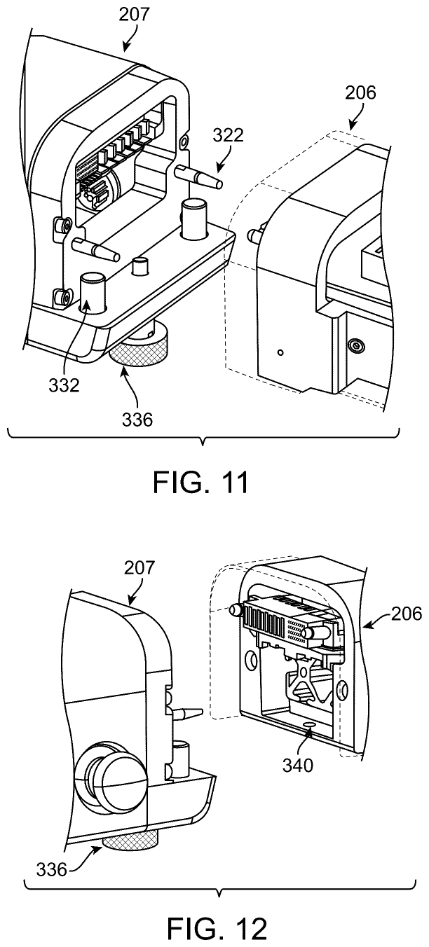

Referring to FIGS. 10-12, in a second embodiment, the secondary fastening mechanism is a spring plunger 336 carried on a proximally extending member 338 of the distal subassembly 207. The spring plunger 336 engages with a bore 340 (FIG. 12) on the horizontal boom assembly 206 of the robot. This interface is referred to herein as the quick release.

The second embodiment also includes features described with respect to the first embodiment. Such features include the circuit boards housed in the distal subassembly and the boom assembly, with connectors that mate together, forming a butt joint, and connect all electrical contacts. The second embodiment further includes the alignment pins 322, compliant elements such as O-rings for allowing the circuit boards to “float,” and safety bolts as the primary fastening mechanism. Each of these features is described with respect to the first embodiment, and so the details will not be repeated here.

By utilizing one central electrical connector and a quick release mechanism, the disclosed configurations will allow a portion of a robotic manipulator to be quickly changed, minimizing operating room downtime and cost to the customer. Although these embodiments make use of quick release features, the robotic manipulator maintains its structural integrity.

1. A surgical robotic manipulator comprising:
a proximal assembly comprising a first plurality of joints operable to move the robotic manipulator in a first plurality of degrees of freedom;
a distal assembly comprising a second plurality of joints operable to move the robotic manipulator in a second plurality of degrees of freedom;
a primary fastening mechanism engaging the distal assembly to the proximal assembly, where the secondary fastening mechanism is a quick release mechanism.

2. The surgical robotic manipulator of claim 1, wherein the secondary fastening mechanism comprises a hook and latch mechanism.

3. The surgical robotic manipulator of claim 2, wherein the secondary fastening mechanism comprises a member on a first one of the proximal assembly and the distal assembly, and the hook and latch mechanism of the other one of the proximal assembly and the distal assembly.

4. The surgical robotic manipulator of claim 1, wherein the secondary fastening mechanism comprises a spring plunger on a first one of the proximal assembly and the distal assembly, and a bore on a second one of the base assembly and the distal assembly, the bore configured to receive a portion of the spring plunger.

5. The surgical robotic manipulator of claim 1, wherein the primary fastening mechanism comprises a plurality of bolts engaging the distal assembly to the proximal assembly.

6. The surgical robotic manipulator of claim 1, further including:
a first circuit board in the distal assembly and a first connector on the first circuit board;
a second circuit board in the base assembly and a second connector on the second circuit board;
wherein the first and second connectors are releasably engaged.

7. The surgical robotic manipulator of claim 1, wherein the proximal assembly includes a vertical column and a horizontal boom, and wherein the primary and secondary fastening mechanisms removably couple the distal assembly to the horizontal boom.

8. The surgical robotic manipulator of claim 1, wherein the proximal assembly is moveable in three degrees of freedom, and the distal assembly is moveable in four degrees of freedom.

9. The surgical robotic manipulator of claim 8, wherein the proximal assembly is moveable in only three degrees of freedom.

10. The surgical robotic manipulator of claim 9, wherein the distal assembly is moveable in only four degrees of freedom.

11. A method of servicing a surgical robotic manipulator, comprising the steps of:
providing a surgical robotic manipulator comprising:
a proximal assembly comprising a first plurality of joints operable to move the robotic manipulator in a first plurality of degrees of freedom,
a distal assembly comprising a second plurality of joints operable to move the robotic manipulator in a second plurality of degrees of freedom,
a primary fastening mechanism securing the distal assembly to the proximal assembly; and
a secondary fastening mechanism engaging the distal assembly to the proximal assembly, wherein the secondary fastening mechanism is a quick release mechanism;
disengaging the primary fastening mechanism;
after disengaging the primary fastening mechanism, disengaging the quick release mechanism;
separating the distal assembly from the proximal assembly;
positioning a replacement distal assembly distal to the proximal assembly and engaging the quick release mechanism to couple the replacement distal assembly to the proximal assembly;
after engaging the quick release mechanism, engaging the primary fastening mechanism.

12. The method of claim 11, further comprising, before disengaging the quick release mechanism, disengaging electrical connectors between the distal assembly and the proximal assembly.

13. The method of claim 11, wherein positioning a replacement distal assembly includes aligning alignment pins of a first one of the replacement distal assembly and the proximal assembly with corresponding bores of a second one of the replacement distal assembly and the proximal assembly, and then inserting said alignment pins into the corresponding bores while advancing at least one of the replacement distal assembly and the proximal assembly towards the other of the replacement distal assembly and the proximal assembly.

14. The method of claim 13, further including, after inserting said alignment pins into the corresponding bores, engaging electrical connectors between the distal assembly and the proximal assembly.
